(19) United States
(12) Patent Application Publication
(10) Pub. No.: US 2020/0315681 A1
(43) Pub. Date: Oct. 8, 2020

(54) APPARATUS AND METHODS FOR PERFORMING A DENTAL COLD SENSITIVITY TEST AND FOR CONTROLLING SWAB SATURATION

(71) Applicant: Illinois Tool Works Inc., Glenview, IL (US)

(72) Inventors: Steven D. Cook, Amarillo, TX (US); Matthew E. Morris, Kennesaw, GA (US); Paul M. Blair, Jonesboro, GA (US); Samantha J. Luedke, Los Angeles, CA (US); Edward P. Vickless, Canton, GA (US); Kenneth D. Caskey, Marietta, GA (US)

(21) Appl. No.: 16/373,384

(22) Filed: Apr. 2, 2019

(51) Int. Cl. A61B 18/02; A61B 5/00; A61C 19/04

(52) U.S. Cl. CPC A61B 18/0218; A61B 5/4824; A61F 13/38; A61B 2018/0231; A61C 19/04

(57) ABSTRACT

The present disclosure is generally directed to a swab and an apparatus for testing dental cold sensitivity. The swab comprises a hollow tube and an absorbent tip fixedly attached to the distal end of the hollow tube. The dispensing apparatus includes a pressurized container and an actuator. The actuator may include a flow control valve to limit the flow of coolant to the absorbent tip or to limit the volume to a metered dose and to prevent the swab from being ejected from the actuator due to excess force.

### FIELD

The present disclosure generally relates to a swab and a coolant dispenser that may be releasably coupled to the swab and configured to control swab saturation. In certain embodiments, a saturated swab may be used for dental cold sensitivity testing.

### BACKGROUND

A common method for testing oral nerve health involves analyzing a patient's reaction to cold. A healthy tooth nerve transmits a cold stimulus. No resulting sense of cold or an intense cold pain, lasting beyond the actual stimulus, can mean the patient has nerve damage. If further testing confirms damage, or devitalization, root treatment may be necessary.

Dentists may test nerve damage by performing a dental cold sensitivity test. Currently, dentists may perform a dental cold sensitivity test by saturating a swab with a coolant, and applying the coolant to a patient's tooth. Spraying the coolant directly at a patient's tooth or teeth can cause undesirable pain and damage. Dentists, therefore, may perform a dental cold sensitivity test by holding a cotton ball or swab with tweezers in one hand, actuating a coolant dispenser with the other hand to dispense coolant until the cotton ball or swab is saturated with coolant, and then applying the saturated cotton ball or swab to the patient's tooth. This process requires dentists to hold a cotton ball or tweezers with one hand and a coolant dispenser with another hand.

Further, because conventional coolant dispensers do not implement any type of flow control valve, they dispense coolant at a rate proportionate to their level of actuation. Without a flow control valve, the user may oversaturate the swab and waste coolant. Further, a straw or tube coupled to the actuator may be “blown out” of (ejected from) the actuator due to excess force when a standard valve is fully actuated.

Thus, there is a need for an improved method for dental cold sensitivity testing that allows a swab to be saturated with coolant (using one hand) without needing to additionally use a pair of tweezers (using another hand) to hold the swab. Further, there is a need for an improved coolant dispenser that, among other things, avoids oversaturation of swabs and inadvertent ejection of straws and tubes.

In addition, current commonly-used coolants can be environmentally unsafe, with relatively high Ozone Depletion Potential (ODP) or Global Warming Potential (GWP) values. For example, each of the cryogenic agents specified in U.S. Pat. No. 5,330,745 have high ODP and GWP values that mean such agents may cause harmful effects in terms of depleting the ozone layer or enhancing global warming.

Thus, there is also a need for coolants that have lower ODP and GWP values.

### SUMMARY

The present disclosure relates generally to a swab and coolant dispenser comprising a mechanism to control swab saturation for performing a dental cold sensitivity test.

According to an aspect of the present disclosure, a swab for performing a dental cold sensitivity test comprises a hollow tube and an absorbent tip. The hollow tube extends from a proximal end to a distal end and defines an internal coolant channel. The absorbent tip is fixedly attached to the distal end of the hollow tube such that the absorbent tip impedes the internal coolant channel at the distal end to prevent coolant from entering or exiting the internal coolant channel without first passing through the absorbent tip. The internal coolant channel is open at the proximal end such that coolant is able to enter or exit the proximal end.

According to another aspect of the present disclosure, a dental cold sensitivity testing apparatus comprises a pressurized container and a swab. The pressurized container contains a coolant. The pressurized container comprises an actuator and an outlet. The swab may be as described above in the previous paragraph. The proximal end of the swab may be releasably coupled to the outlet (for example, by inserting the proximal end of the swab into the outlet). When a user actuates the actuator, a volume of coolant may be dispensed such that it passes through the outlet, the proximal end, and the internal coolant channel to reach the absorbent tip fixedly attached to the distal end of the swab. The absorbent tip may be at least substantially saturated with the volume of coolant.

According to another aspect of the present disclosure, a user may use the swab and dental cold sensitivity testing apparatus described in the above paragraphs to perform a dental cold sensitivity test. The user may releasably couple the proximal end of the swab to the outlet. Next, the user may actuate the actuator to cause a volume of coolant to pass through the outlet, the proximal end, and the internal coolant channel to reach the absorbent tip fixedly attached to the distal end of the swab. The user may thus at least substantially saturate the absorbent tip with the volume of coolant.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

The following is a description of the examples depicted in the accompanying drawings. The figures are not necessarily to scale, and certain features and certain views of the figures may be shown exaggerated in scale or in schematic in the interest of clarity or conciseness.

FIG. 1 shows a schematic cross-sectional view of a swab for performing a dental cold sensitivity test according to an embodiment of the present disclosure.

FIG. 2A shows a schematic view of a coolant dispenser releasably coupled to a swab for performing a dental cold sensitivity test according to an embodiment of the present disclosure.

FIG. 2B shows a schematic view of a coolant dispenser releasably coupled to a swab for performing a dental cold sensitivity test according to another embodiment of the present disclosure.

FIG. 3A shows a schematic cross-sectional view of a swab for performing a dental cold sensitivity test releasably coupled to an actuator according to an embodiment of the present disclosure.

FIG. 3B shows a schematic view of a swab for performing a dental cold sensitivity test releasably coupled to an actuator according to another embodiment of the present disclosure.

FIG. 4 shows a schematic cross-sectional view of a flow control valve for a coolant dispenser according to an embodiment of the present disclosure.

FIG. 5 shows a schematic view of a swab for performing a dental cold sensitivity test according to another embodiment of the present disclosure.

The foregoing summary, as well as the following detailed description, will be better understood when read in conjunction with the figures. It should be understood that the claims are not limited to the arrangements and instrumentality shown in the figures. Furthermore, the appearance shown in the figures is one of many ornamental appearances that can be employed to achieve the stated functions of the apparatus.

### DETAILED DESCRIPTION

In the following detailed description, specific details may be set forth in order to provide a thorough understanding of embodiments of the present disclosure. However, it will be clear to one skilled in the art when disclosed examples may be practiced without some or all of these specific details. For the sake of brevity, well-known features or processes may not be described in detail. In addition, like or identical reference numerals may be used to identify common or similar elements.

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

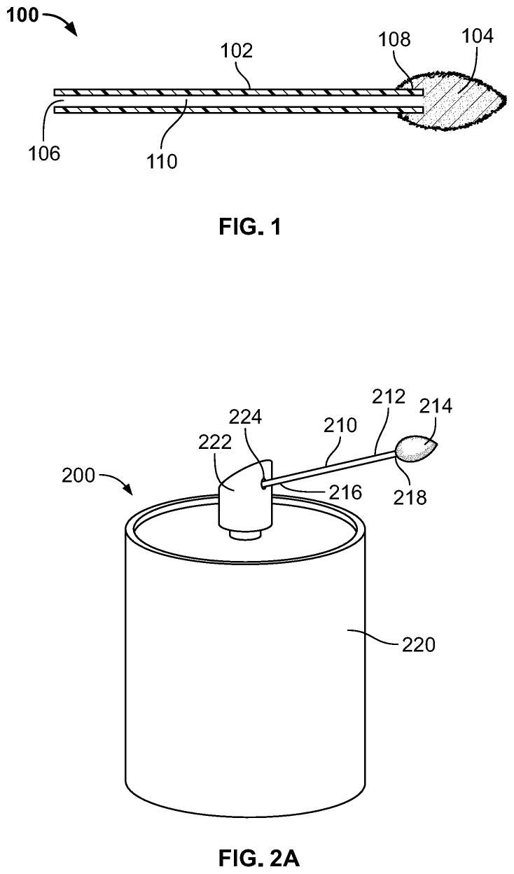

FIG. 1 shows a swab 100 for performing a dental cold sensitivity test according to the present disclosure. The swab 100 includes a hollow tube 102 and an absorbent tip 104. The hollow tube 102 extends from a proximal end 106 to a distal end 108 and defines an internal coolant channel 110 such that fluids may pass through the hollow tube 102 unobstructed until such fluids reach the absorbent tip 104. The absorbent tip 104 may be fixedly attached to the distal end 108 such that the absorbent tip 104 impedes the internal coolant channel 110 at the distal end 108 to prevent coolant from entering or exiting the internal coolant channel 110 without first passing through the absorbent tip 104. The internal coolant channel 110 may be open at the proximal end 106 such that coolant is able to enter or exit the proximal end 106.

The hollow tube 102 may be made of any suitable material, including plastics such as polystyrene (PS), polyethylene terephthalate (PET), or polypropylene (PP), or metal such as aluminum. The hollow tube 102 may be transparent or opaque. The absorbent tip 104 may be made of any suitable material, including fibrous materials such as synthetic fibers and natural fibers (including loosely woven cotton), foam materials, or a combination thereof. The absorbent tip 104 may be attached to the hollow tube 102 by any suitable method, including heat sealing, ultrasound, radio frequency (RF), thermally, adhesive, and solvent bonding. According to one aspect of the present disclosure, a user may use the swab 100 to test dental sensitivity by first saturating or substantially saturating the absorbent tip 104 with a coolant and then applying the at least substantially saturated absorbent tip 104 to an exposed area in an oral cavity, including a tooth or gum line. In certain embodiments, the swab 100 may be reusable. In other embodiments, the swab 100 may be disposable. According to the present disclosure, a saturated absorbent tip may be defined as an absorbent tip that is unable to absorb any more coolant. For example, one may be able to infer that a swab is saturated if the swab visibly appears to be saturated with coolant or if coolant begins to drip from the swab. A substantially saturated absorbent tip may be defined as an absorbent tip that is approaching saturation. Depending on the coolant being absorbed, a substantially saturated absorbent tip may be, for example, at least 80% saturated, at least 90% saturated, at least 95% saturated with coolant. According to certain embodiments of the present disclosure, the coolant may be a cryogenic agent. According to other embodiments of the present disclosure, the coolant may be any fluid, including water, acid, or a chemical solution.

The swab 100 for performing a dental cold sensitivity test may be any suitable length. Similarly, the absorbent tip 104 may be any suitable size. For example, the length of the absorbent tip 104 may vary upon the size of a tooth being tested for dental sensitivity. According to certain embodiments, approximately 1/3 of a tooth's surface may be exposed to the absorbent tip 104. According to certain embodiments, the absorbent tip 104 may be between ¼ and ½ inches long.

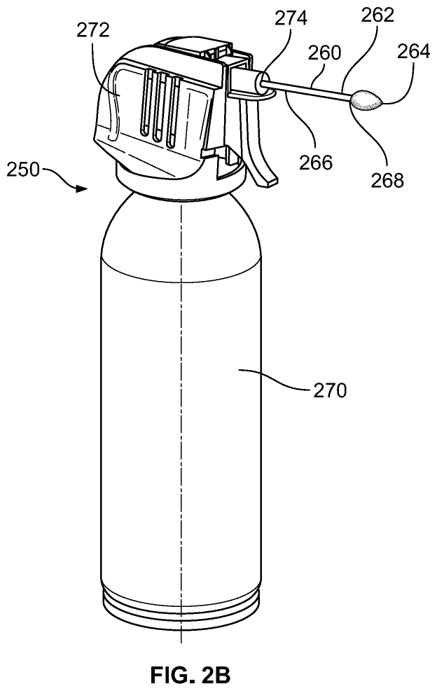

FIG. 2A shows a dental cold sensitivity testing apparatus 200 according to the present disclosure. The dental cold sensitivity testing apparatus 200 comprises a swab 210, a pressurized container 220, an actuator 222, and an outlet 224. The swab 210 may be as described above, and may thus have a hollow tube 212 that extends from a proximal end 216 to a distal end 218 and defines an internal channel. An absorbent tip 214 may be fixedly attached to the distal end 218. The proximal end 216 of the swab 210 may be releasably coupled (directly or indirectly) to the outlet 224. For example, the proximal end 216 may be inserted into the outlet 224. In certain embodiments, the outlet 224 may be part of the actuator 222. In other embodiments, the outlet 224 may be separate from the actuator 222. In certain embodiments, the actuator 222 may comprise a valve, and the actuator 222 may be actuated by pushing down on the actuator 222. In such embodiments, the outlet 224 may be a nozzle housed within the actuator 222. When a user actuates the actuator 222, a volume of coolant stored within the pressurized container 220 is dispensed. The volume of coolant passes through the outlet 224, through the internal channel of the hollow tube 212, and into the absorbent tip 214.

FIG. 2B shows an alternative embodiment of a dental cold sensitivity testing apparatus 250 according to the present disclosure. The dental cold sensitivity testing apparatus 250 comprises a swab 260, a pressurized container 270, an actuator 272, and an outlet 274. The swab 260 may be as described above, and may thus have a hollow tube 262 that extends from a proximal end 266 to a distal end 268 and defines an internal channel. An absorbent tip 264 may be fixedly attached to the distal end 268. The proximal end 266 of the swab 260 may be releasably coupled (directly or indirectly) to the outlet 274. When a user actuates the actuator 272, a volume of coolant stored within the pressurized container 270 is dispensed. The volume of coolant passes through the outlet 274, through the internal channel of the hollow tube 262, and into the absorbent tip 264.

A suitable length of time needed to dispense coolant in order to saturate or substantially saturate an absorbent tip 214 may vary depending on such factors as the coolant that is used, the level of actuation of the actuator 222, the typical flow rate of coolant being dispense by the actuator, and the size of the absorbent tips. For the majority of coolants, actuators, and absorbent tips that may be used according to the present disclosure, dispensing the coolant for between 1 and 15 (or between 3 and 10) seconds will saturate or substantially saturate an absorbent tip 214.

According to one aspect of the present disclosure, the actuator 222 may be configured to dispense coolant at a maximum flow rate. For example, the actuator 222 may comprise a flow control valve configured to dispense coolant at a flow rate no greater than a specified limit. For example, this limit could be no greater than 100 microliters per second (μL/s). By limiting the maximum flow rate, one can prevent a swab 210 from being “blown out” or ejected from an outlet 224 during actuation of the actuator 222.

According to another aspect of the present disclosure, the actuator 222 may be configured to dispense coolant at a constant flow rate regardless of the level of actuation. For example, the actuator 222 may comprise a flow control valve configured to dispense coolant at a given flow rate once the actuator 222 is actuated beyond a threshold level of actuation.

According to another aspect of the present disclosure, the actuator 222 may be configured to dispense coolant at a variable flow rate, where the flow rate depends on the actuator's level of actuation. For example, the actuator 222 may comprise a valve configured to dispense coolant at a greater flow rate as the actuator 222 is further actuated (e.g., as more pressure is applied to the actuator 222, coolant is dispensed at a greater flow rate). When the user actuates the actuator 222, the coolant dispenses. When the user stops actuating the actuator 222, the coolant stops dispensing.

According to another aspect of the present disclosure, the actuator 222 may be configured to dispense a metered dose (i.e., a specific volume) of coolant regardless of the level of actuation. For example, the actuator 222 may comprise a valve configured to dispense a specific volume of coolant once the actuator 222 is actuated beyond a threshold level of actuation. According to certain aspects of the present disclosure, this metered dose or specific volume may be between 25 and 1000 microliters (μL), between 30 and 300 μL, or between 40 and 180 μL. For example, this metered dose may be 50 μL, 60 μL, 75 μL, 80 μL, 90 μL, 95 μL, 100 μL, 110 μL, 120 μL, or 125 μL.

According to the present disclosure, a dental cold sensitivity testing apparatus 200 may be used to test dental sensitivity. To do so, a user may first releasably couple the proximal end 216 of the swab 210 to the outlet 224. Next, the user may actuate the actuator 222 to cause a volume of coolant to pass through the outlet 224, the proximal end 216, and the internal coolant channel to reach the absorbent tip 214 fixedly attached to the distal end 218 of the swab 210. In doing so, the user may saturate or substantially saturate the absorbent tip 214 with the volume of coolant. The saturation or near-saturation of the absorbent tip 214 may occur due to the level of actuation (e.g., from the user actuating the actuator 222 for a sufficiently long time at a sufficient pressure to reach saturation or near-saturation). In embodiments where the dental cold sensitivity testing apparatus 200 comprises a flow control valve, the level of saturation may be controlled by the flow control valve (independent of the level of actuation beyond a threshold amount needed to commence actuation). In certain scenarios, a user may need to actuate the actuator 222 more than once (for example, two or three separate times) in order to achieve full or substantial saturation. After fully or substantially saturating the absorbent tip 214, the user may decouple the swab 210 from the outlet 224 and, holding the swab 210 away from the distal end 218, apply the saturated absorbent tip 214 to a particular site (such as an exposed area of an oral cavity to test dental sensitivity). For example, the exposed area of the oral cavity may be a tooth, gum, or dental pulp. In other scenarios, the particular site may be away from an oral cavity and may be, for example, a skin lesion (for example, the saturated absorbent tip 214 could be applied to a wart in order to remove the wart).

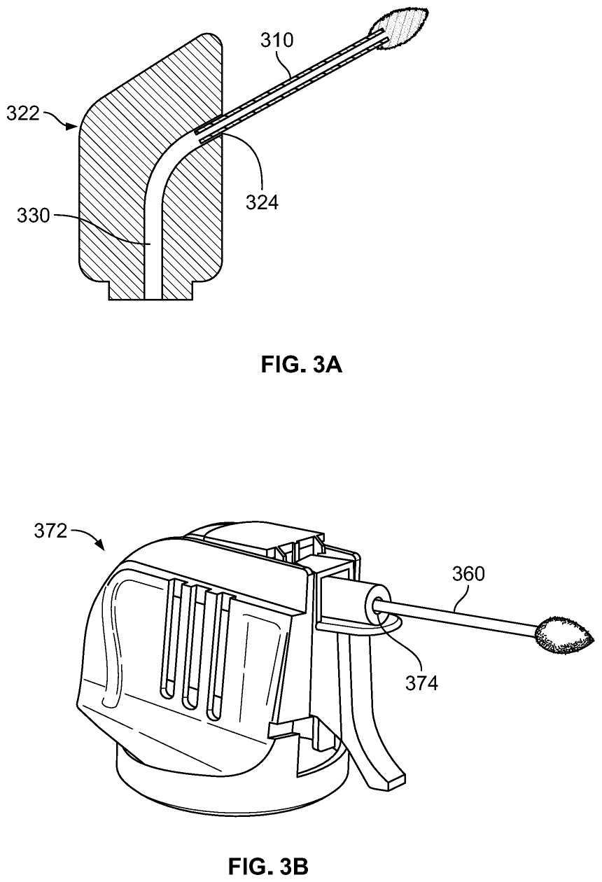

FIG. 3A shows an example of how a hollow tube 310 may be releasably coupled to an outlet 324 located in an actuator 322 according to the present disclosure by inserting the hollow tube 310 into the outlet 324. As shown in this embodiment, the outlet 324 may be part of an internal channel 330 in the actuator 322.

FIG. 3B shows an alternative embodiment of an example of how a hollow tube 360 may be releasably coupled to an outlet 374 located in an actuator 372 according to the present disclosure by inserting the hollow tube 360 into the outlet 374.

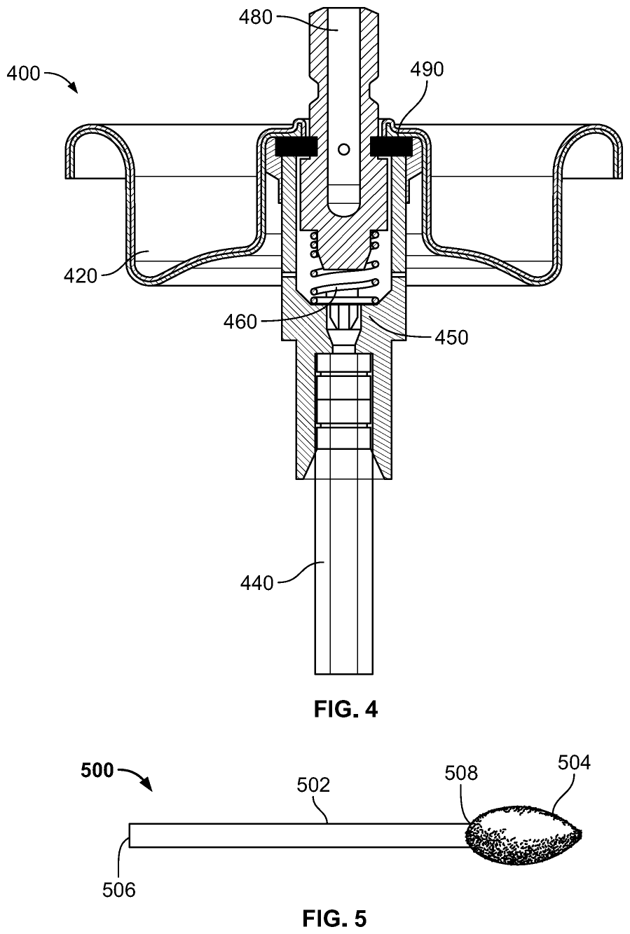

FIG. 4 shows an example of a flow control valve 400 that may be used in conjunction with an actuator (not shown in this figure) according to the present disclosure. As shown in FIG. 4, a stem 480 may be housed inside a housing 450. The base of the stem may be surrounded by a stem gasket 490, which provides sealing of the valve. A spring 460 may be fitted inside the housing 450 to provide a resilient force against the stem 480 to keep the valve closed when not in operation. A dip tube 440 may be fluidly connected to the housing 450. The stem 480 and housing 450 may be mounted in a mounting cup 420. When a user depresses or actuates the actuator (placed on the stem) by pressing on the stem 480, coolant may be dispensed via the dip tube 440, housing 490, stem 480, and actuator. The shape of the stem 480 and housing 450 acts as a flow control valve that prevents coolant from being dispensed at too great of a flow rate.

According to another embodiment of the present disclosure, FIG. 5 shows a swab 500 with a stem 502 having a proximal end 506 and a distal end 508. According to this embodiment, the stem 502 is not hollow. An absorbent tip 504 may be fixedly attached to the distal end 508 of the stem 502. When using this swab 500, a user may saturate the swab 500 without attaching it to a coolant dispenser. A user may do this by, for example, holding the swab 500 in one hand in front of an outlet, and actuating the actuator with the other hand until the swab is saturated (or at least substantially saturated). The saturated absorbent tip 504 may then be applied to an exposed area in an oral cavity to test dental sensitivity.

According to the present disclosure, the coolant used may be selected from the following: R-1234yf (HFO-1234yf) 2,3,3,3-Tetrafluoropropene, R-1234ze (HFO 1234ze) trans-1,3,3,3-Tetrafluoroprop-1-ene CF₃CH═CHF, cis-1,3,3,3-Tetrafluoroprop-1-ene, CF₃CH═CHF R-32 1,1,1-Chlorodifluoromethane, R-744 CO2, R-514A, (HFO-1336mzzZ) trans-1,1,1,4,4,4-Hexafluoro-2-butene/trans-1,2-dichloroethylene, and R-1233zd(E) trans-1-chloro-3,3,3-trifluoropropene, (HFC 134a) 1,1,1,2 Tertrafluoroethane, (HFC 152a) 1,1 difluoroethane, or a mixture thereof. These coolants are also shown in Table 1. The coolants disclosed herein are more environmentally sustainable when compared to other coolants conventionally in use. The relative environmental suitability of a coolant may be quantified by its Global Warming Potential (GWP) and Ozone Depletion Potential (ODP). GWP is a relative measurement of the amount of heat a greenhouse gas traps in the atmosphere as compared to a similar mass of carbon dioxide over a specific time interval. GWP is expressed as a factor of the standardized GWP of carbon dioxide (1.0). A high GWP correlates with large infrared absorption and long atmospheric life, and as such represents the potential of a substance to contribute to global warming. ODP is a relative measurement of the amount of ozone layer degradation a coolant can cause as compared to the standardized ODP of trichlorofluoromethane (R-11 or CFC-11), where trichlorofluoromethane has an ODP of 1.0. A higher ODP correlates with a substance expected to cause greater ozone layer degradation. The coolants disclosed herein in the present disclosure have low GWP and no ODP.

TABLE 1

| Coolant | ODP | GWP |
|---|---|---|
| R-1234yf (HFO-1234yf) 2,3,3,3- | 0 | <1 |
| Tetrafluoropropene | | |
| R-1234ze (HFO 1234ze) trans-1,3,3,3- | 0 | <1 |
| Tetrafluoroprop-1-ene CF₃CH═CHF | | |
| R-32 - 1,1,1-Chlorodifluoromethane | 0 | >2 |
| R-744 - CO₂ | 0 | 1 |
| R-514A, (HFO-1336mzzZ) trans-1,1,1,4,4,4- | 0 | 2 |
| Hexafluoro-2-butene/trans-1,2- | | |
| dichloroethylene | | |
| R-1233zd(E) trans-1-chloro-3,3,3- | 0 | <1 |
| trifluoropropene | | |
| R-134a - 1,1,1,2 Tetrafluoroethane | 0 | 1300 |
| (HFC 152a) - 1,1 diflouroethane | 0 | 150 |
| 1,1,1,3,3-Pentafluoropropane (HFC-245fa) | 0 | 950 |

The various aspects and embodiments disclosed herein are not intended to be limiting. Other embodiments may be utilized, and other changes may be made, without departing from the spirit or scope of the subject matter presented herein. It will be readily understood that the aspects of the present disclosure, as generally described herein and illustrated in the figures, can be arranged, substituted, combined, separated, and designed in a wide variety of different configurations, all of which are contemplated herein.

1. A swab for performing a dental cold sensitivity test comprising:
a hollow tube extending from a proximal end to a distal end and defining an internal coolant channel; and
an absorbent tip fixedly attached to the distal end of the hollow tube such that the absorbent tip impedes the internal coolant channel at the distal end to prevent coolant from entering or exiting the internal coolant channel without first passing through the absorbent tip,
wherein the internal coolant channel is open at the proximal end such that coolant is able to enter or exit the proximal end.

2. The swab of claim 1, wherein the absorbent tip comprises a fibrous material, a foam material, or a mixture of fibrous and foam materials.

3. The swab of claim 1, wherein the absorbent tip comprises loosely woven cotton.

4. The swab of claim 1, wherein the absorbent tip is fixedly attached to the distal end of the hollow tube by heat sealing.

5. The swab of claim 1, wherein the swab is configured to test dental sensitivity by applying the swab to an exposed area in an oral cavity.

6. A dental cold sensitivity testing apparatus comprising:
a pressurized container containing a coolant,
wherein the pressurized container comprises an actuator and an outlet;
wherein the proximal end of the swab of claim 1 is releasably coupled to the outlet;
wherein when a user actuates the actuator, a volume of coolant is dispensed such that it passes through the outlet, the proximal end, and the internal coolant channel to reach the absorbent tip fixedly attached to the distal end of the swab; and
wherein the absorbent tip is at least substantially saturated with the volume of coolant.

7. The dental cold sensitivity testing apparatus of claim 6, further comprising a flow control valve configured to limit the flow rate of the coolant such that when a user actuates the actuator, the coolant cannot be dispensed at a flow rate greater than a specified limited flow rate.

8. The dental cold sensitivity testing apparatus of claim 6, wherein the dental cold sensitivity testing apparatus is configured to dispense coolant at a constant flow rate when a user actuates the actuator, regardless of the actuator's level of actuation.

9. The dental cold sensitivity testing apparatus of claim 6, wherein the dental cold sensitivity testing apparatus is configured to dispense coolant at a variable flow rate that depends upon the actuator's level of actuation.

10. The dental cold sensitivity testing apparatus of claim 6, wherein the dental cold sensitivity testing apparatus is configured to dispense a metered dose of coolant at a specific volume regardless of the actuator's level of actuation.

11. The dental cold sensitivity testing apparatus of claim 10, wherein the specific volume of the metered dose is between 25 μL and 125 μL.

12. The dental cold sensitivity testing apparatus of claim 6, wherein the coolant is selected from the group consisting of: R-1234yf (HFO-1234yf) 2,3,3,3-Tetrafluoropropene; R-1234ze (HFO 1234ze) trans-1,3,3,3-Tetrafluoroprop-1-ene CF₃CH═CHF; R-32-1,1,1-Chlorodifluoromethane; R-744-CO2; R-514A, (HFO-1336mzzZ) trans-1,1,1,4,4,4-Hexafluoro-2-butene/trans-1,2-dichloroethylene; R-1233zd(E) trans-1-chloro-3,3,3-trifluoropropene; R-134a-1,1,1,2 Tetrafluoroethane; (HFC 152a)-1,1 diflouroethane; 1,1,1,3,3-Pentafluoropropane (HFC-245fa) or a mixture thereof.

13. The dental cold sensitivity testing apparatus of claim 6, wherein the dental cold sensitivity apparatus is configured to test dental sensitivity by applying the swab to an exposed area in an oral cavity.

14. A method for testing dental cold sensitivity comprising the steps of:
a. providing a swab having:
a hollow tube extending from a proximal end to a distal end and defining an internal coolant channel, and
an absorbent tip fixedly attached to the distal end of the hollow tube such that absorbent tip impedes the internal coolant channel at the distal end to prevent coolant from entering or exiting the internal coolant channel without first passing through the absorbent tip,
wherein the internal coolant channel is open at the proximal end such that coolant is able to enter or exit the proximal end;
b. providing a pressurized container containing a coolant and comprising an actuator and an outlet;
c. inserting the proximal end of the swab into the outlet of the pressurized container;
d. actuating the actuator to cause a volume of coolant to pass through the outlet, the proximal end, and the internal coolant channel to reach the absorbent tip fixedly attached to the distal end of the swab; and
e. at least substantially saturating the absorbent tip with the volume of coolant.

15. The method of claim 15, wherein the pressurized container further comprises a flow control valve configured to limit the flow rate of the coolant such that during step (d), the coolant cannot be dispensed at a flow rate greater than a specified limited flow rate.

16. The method of claim 15, wherein the pressurized container further comprises a flow control valve configured to control the flow rate of the coolant such that during step (d), the coolant is dispensed a constant flow rate regardless of the user's actuation or throttling of the actuator.

17. The method of claim 15, wherein the pressurized container further comprises a flow control valve configured to control the flow rate of the coolant such that during step (d), the coolant is dispensed a variable flow rate depending on the user's actuation or throttling of the actuator.

18. The method of claim 15, wherein the pressurized container further comprises a valve configured to dispense a metered dose of coolant, such that during step (d), a fixed volume of coolant is dispensed.

19. The method of claim 15, wherein the coolant is selected from the group consisting of: R-1234yf (HFO-1234yf) 2,3,3,3-Tetrafluoropropene; R-1234ze (HFO 1234ze) trans-1,3,3,3-Tetrafluoroprop-1-ene CF₃CH═CHF; R-32-1,1,1-Chlorodifluoromethane; R-744-CO₂; R-514A, (HFO-1336mzzZ)trans-1,1,1,4,4,4-Hexafluoro-2-butene/trans-1,2-dichloroethylene; R-1233zd(E) trans-1-chloro-3,3,3-trifluoropropene; R-134a-1,1,1,2 Tetrafluoroethane; (HFC 152a)-1,1 diflouroethane; 1,1,1,3,3-Pentafluoropropane (HFC-245fa) or a mixture thereof.

20. The method of claim 15, further comprising a step (f) of testing dental sensitivity by applying the swab to an exposed area in an oral cavity.
